(19) United States
(12) Patent Application Publication
(10) Pub. No.: US 2014/0186927 A1
(43) Pub. Date: Jul. 3, 2014

(54) Process for the Production and Utilization of Chlamydospore Rich Slurry Inoculum

(71) Applicants: Jacob Michael Winiski, Troy, NY (US); Sue Sweet Van Hook, Cambridge, NY (US); Gavin McIntyre, Troy, NY (US)

(72) Inventors: Jacob Michael Winiski, Troy, NY (US); Sue Sweet Van Hook, Cambridge, NY (US); Gavin McIntyre, Troy, NY (US)

(21) Appl. No.: 14/099,416

(22) Filed: Dec. 6, 2013

Related U.S. Application Data
(60) Provisional application No. 61/748,209, filed on Jan. 2, 2013.

(51) Int. Cl. C12N 3/00

(52) U.S. Cl. CPC C12N 3/00
USPC 435/242; 435/256.8

(57) ABSTRACT

The process for the production of a chlamydospore rich slurry inoculum begins with a substrate colonized with a desired Basidiomycete fungus capable of producing chlamydospores during vegetative growth. The colonized substrate is treated to increase the chlamydospore production and content in said spawn and thereafter combined with water at rate of at least 1:6 spawn:water to obtain a slurry inoculum. The inoculum may then be agitated to populate a water fraction with chlamydospores or macerated to homogenously distribute the chlamydospores. Soaking of the agitated/macerated inoculum for a time sufficient to further stimulate production of chlamydospores via water shock and obtain a chlamydospore rich slurry

This application claims the benefit of Provisional Patent Application 61/748,209 filed Jan. 2, 2013.

This invention relates to a process for the production of a chlamydospore rich slurry inoculum.

### BACKGROUND

A chlamydospore is a large, thick-walled, asexual resting spore produced within the mycelium of many fungi. The primary function of a chlamydospore is one of survival—surviving conditions unfavorable to mycelial growth—not dispersal (as with sexual spores). A chlamydospore can be formed as a part of normal vegetative growth or in response to specific environmental conditions (for example desiccation, heat, cold, water shock). Chlamydospores are typically formed in dikaryotic mycelium (and sometimes monokaryotic) and contain nuclei from each parental type.

For example, there is a traditional grain spawn inoculation technique. In this technique, pre-colonized grain particles are distributed through a substrate intended for colonization by a given fungus, followed by mycelium growing (or “jumping”) from the grain particles onto the substrate. Typically, the grain particles are distributed throughout the substrate and mycelial growth occurs.

Media and processes for generating and isolating chlamydospores have been explored and published (see EP 1270717A1 and US 2008/0264858) primarily in relation to Trichoderma spp. and other ascomycetes in the area of pest management.

Identification of the relative presence or absence of chlamydospores in higher Basidiomycetes is a common taxonomic tool, but no functional applications for chlamydospores within the order Polyporales currently exist, nor do processes which utilize chlamydospores to expand and bolster the effectiveness of existing inoculation methods.

As a significant number of species within Polyporales are of economic importance (food, medicinal and material purposes), a process leveraging the specific asexual spore morphology described here would have the potential for significant industrial benefit.

Spore mass inoculation, specifically utilizing sexual spores derived via soaking the organism's fruit body in water to solicit sporulation and induce germination, has been explored. This process requires the organism to mature and produce fruit bodies in order to collect the sexual spores. Because this process utilizes sexual reproduction and genetic recombination, the process does not allow for the utilization and maintenance of a single strain/genetic individual.

Common mushroom and fungal cultivation procedures call for chemical or heat treatment of any substrate intended for inoculation with, and cultivation of, a given fungus. Typically this is done with either pasteurization or sterilization to reduce/eliminate bioburden (the relative amount of microbial life present) in the substrate intended for inoculation. A typical sterilization procedure would consist of heating the substrate to a temperature above 250 F for a period of 1 hour. This process requires sterilization equipment, energy for heat, and adequate time for heating and cooling to occur prior to inoculation. Further, after sterilization aseptic handling is required to reduce chances of introducing contaminant organisms, which requires special equipment and training (laminar flow hoods, sterile equipment, consumable PPE) to perform properly. The need for aseptic procedure can be reduced with pasteurization of substrate—purposeful cultivation of thermophilic bacteria by heating the substrate between 140-180 F for at least one hour—but special equipment, energy, and time is still required.

### Objects of the Invention

It is an object of the invention to derive asexual chlamydospores from a mother vegetative mycelium and to then use both the mother mycelium and asexual chlamydospores as an inoculant concurrently.

It is another object of the invention to leverage the dispersal and germination of chlamydospores during common inoculation procedures (i.e. dispersal of spawn consisting of particles of substrate pre-colonized with a fungus through virgin substrate).

It is another object of the invention to obtain an increased growth rate of fungus through a substrate as compared to traditional inoculation techniques via distribution and germination of chlamydospores.

It is another object of the invention to obtain a higher concentration of chlamydospores in a mother mycelium prior to inoculation.

It is another object of the invention to be able to use non-traditional lignocellulose substrates as spawn via the production, distribution and germination of chlamydospores.

### BRIEF DESCRIPTION OF THE INVENTION

Briefly, the invention provides a process for the production of chlamydospores and, in particular, a process for optimizing concentrations and distribution of chlamydospores during inoculation of a solid or liquid substrate with a given fungus. Preferably, the fungus is a Basidiomycete species within the order Polyporales that produces significant quantities of chlamydospores during primary vegetative growth or under specific environmental or nutritive conditions.

Chlamydospores are liberated from the grain particles prior to inoculation and are distributed with the grain spawn, populating spaces between grain particles. After inoculation, the chlamydospores germinate and germ tubes begin extending from the chlamydospores concurrently with growth from the grain particles, eventually forming a cohesive mycelial network.

These and other objects and advantages of the invention will become more apparent from the following detailed description taken in conjunction with the accompanying drawings wherein:

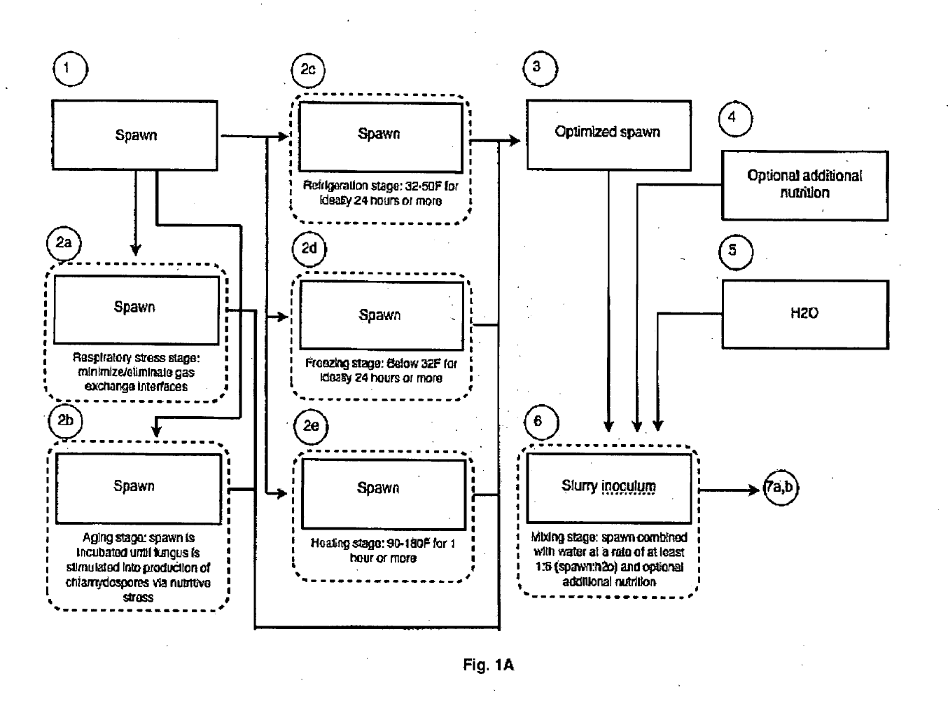

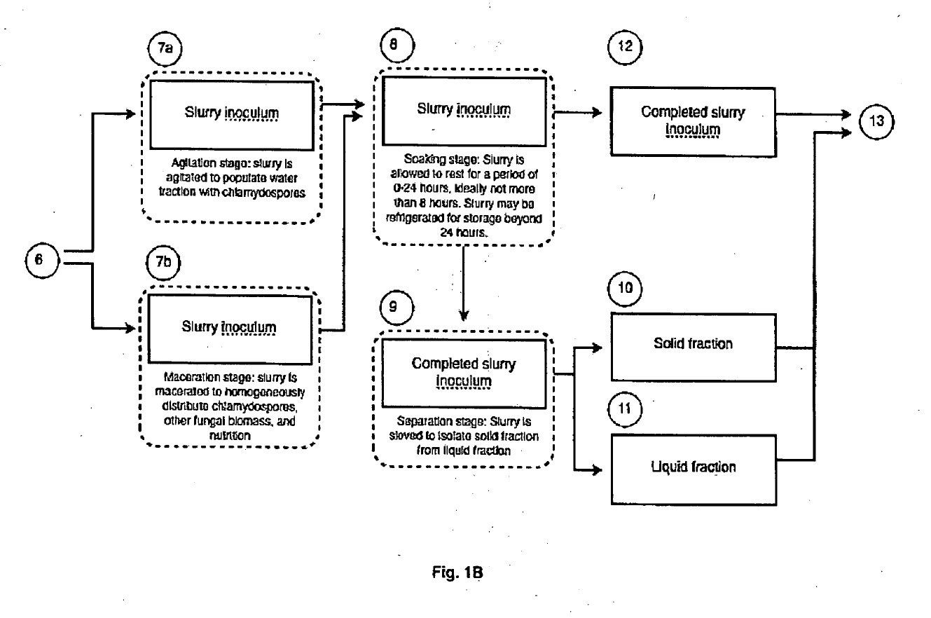

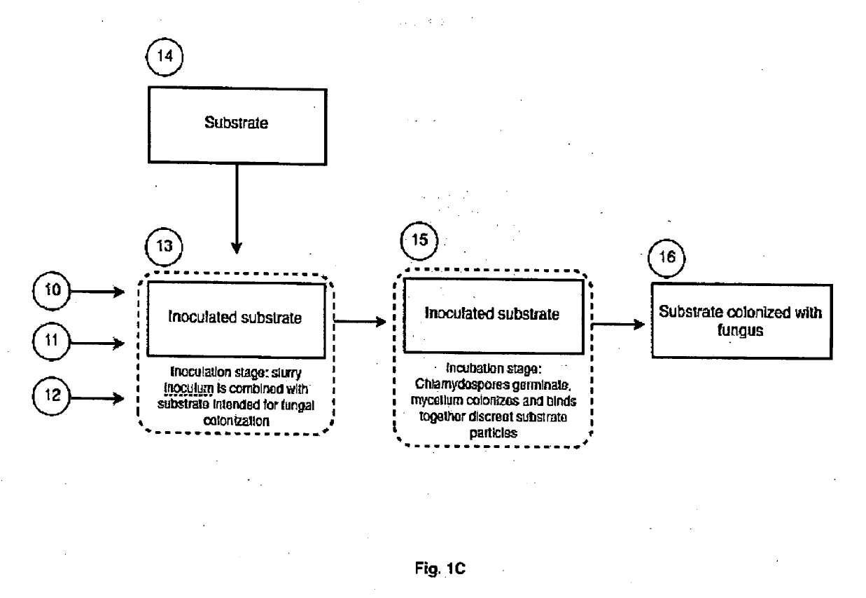

FIGS. 1a to 1c illustrate a flow diagram of a process in accordance with the invention;

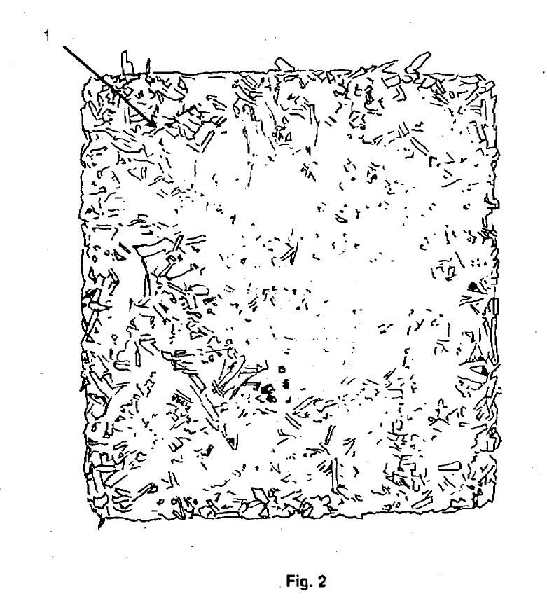

FIG. 2 pictorially illustrates a grain:H₂O+xylose slurry inoculated substrate in accordance with the invention;

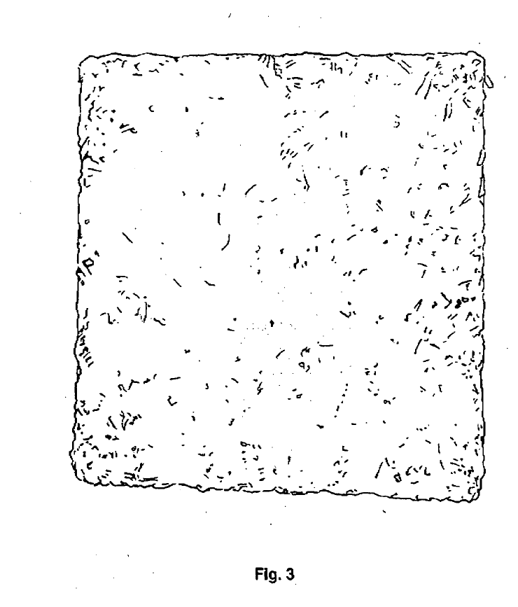

FIG. 3 pictorially illustrates a grain:H₂O+xylose+SBY slurry inoculated substrate in accordance with the invention;

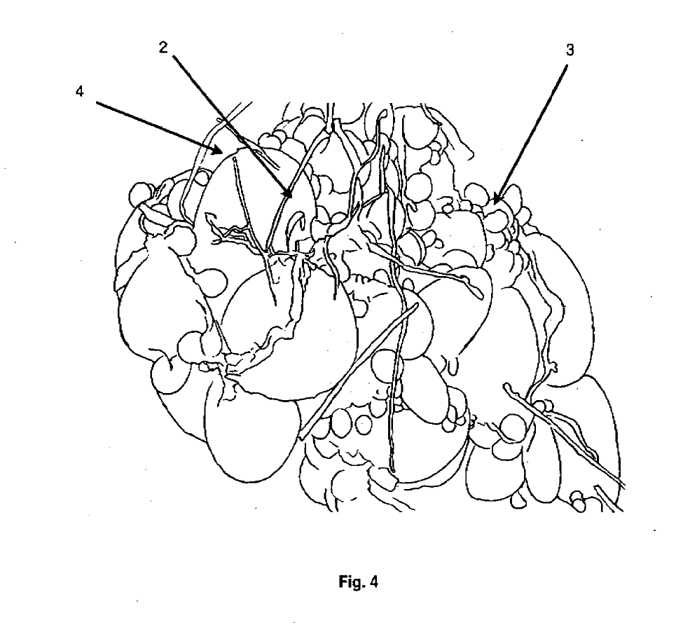

FIG. 4 pictorially illustrates an SEM imaging of chlamydospores produced by a Daedaleopsis spp. after aging in accordance with the invention; and

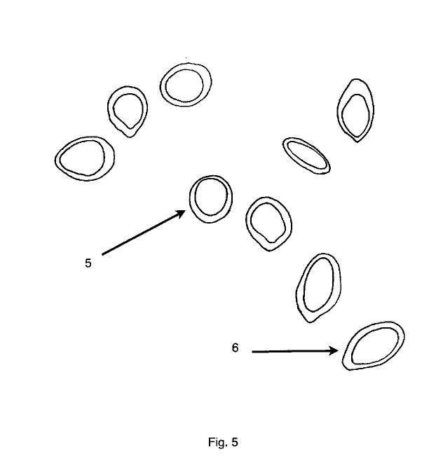

FIG. 5 illustrates a light microscope image of chlamydospores produced in accordance with the invention.

Referring to FIGS. 1a to 1c, in one embodiment, the process comprises the following steps:

1) Obtain spawn consisting of one of the following substrates colonized with the desired Basidiomycete fungus:
A) Grain (examples: millet, rye, or other cereal grain)
B) Lignocellulose (examples: cereal straw, corn stover, hardwood sawdust)
C) Ideally any of the above further optimized with at least 3% fat, 20% starch, and 10% protein

2) Perform any combination of the following to increase chlamydospore production and content in spawn. The presence and concentration of chlamydospores can be verified via light microscope and hemocytometer.
a) Respiratory stress stage—Place spawn in a container without gas exchange interfaces, placing the fungus under respiratory stress, leading to stimulation of chlamydospore formation.
b) Aging stage—spawn is incubated for an extended period of time until fungus is stimulated into production of chlamydospores via nutritive stress.
c) Refrigeration stage—Place spawn in refrigeration between 32-50° F. for ideally at least 24 hours or more.
d) Freezing stage—Place spawn in freezer below 32° F. for ideally at least 24 hours or more.
e) Heating stage—heat spawn to between 90° F. and 180° F. for at least one hour or more.

3) obtain optimized spawn.

4) add any combination of optional additional nutrients (as noted below) to the optimized spawn.
A) No more than 5 g/L carbon 6 monosaccharide or oligosaccharide (such as glucose or maltodextrin)
B) 5-20 g/L carbon 5 monosaccharide (such as xylose)
C) At least 50 ml/L yeast nutrient (such as spent brewer's yeast)

5) Add water to the optimized spawn at a rate of at least 1:6 spawn:water, ideally 1:12

6) Mixing stage—spawn combined with water and optional additional nutrient.

7) Perform one or both of the below steps with slurry inoculum produced during steps 1-6
a) Agitation stage—slurry is agitated with swirling, stirring, or shaking to release chlamydospores from solid (spawn) fraction of slurry and to populate water fraction with chlamydospores.
b) Maceration stage—slurry is macerated (Ideally mechanically) to homogeneously distribute chlamydospores, other fungal biomass, and nutrition provided by spawn into water fraction of slurry

8) Soaking stage—slurry is allowed to rest for a period of 0-24 hours, ideally for no more than 8 hours, to further stimulate production of chlamydospores via water shock. The resultant chlamydospore rich slurry may then be refrigerated for storage beyond 24 hours if required.

9) Separation stage—completed chlamydospore rich slurry inoculum is sieved to isolate the solid fraction from the liquid (water) fraction.

13) Inoculation stage—the separated solid fraction (10) and the separated liquid fraction (11) or the completed slurry inoculum (12) from step 8 can be combined with a substrate (14) intended for fungal colonization. Mix thoroughly to disburse chlamydospore rich slurry inoculum throughout substrate

The substrate intended for inoculation may be prepared for inoculation by either:
a) Combining with desired moisture and nutrition content and heat pasteurized/sterilized
b) Process of A without any heat pasteurization/sterilization or other treatment intended to reduce the microbial bioburden of the substrate prior to inoculation

15) Incubation stage—Incubate inoculated substrate in an environment appropriate for the species of fungus being cultivated until chlamydospores germinate, germ tubes expand into mycelium, and:
a) the mycelium colonizes and binds together discrete particles of substrate, or
b) Other standards for colonization have been met

16) Final stage—the substrate is colonized with the fungus.

The process of the invention increases the colonization rate, decreases the contamination rate, and decreases consumption of costly spawn via methods for optimizing 1) production of chlamydospores by the given Basidiomycete fungus, 2) distribution of chlamydospores during inoculation of solid or liquid substrate, 3) treatments for increasing competitiveness of a given fungus with other microorganisms.

The process leverages the dispersal and germination of chlamydospores during common inoculation procedures (i.e. dispersal of spawn consisting of particles of substrate pre-colonized with a fungus through virgin substrate) by utilizing either A) a species of higher Basidiomycete that naturally produce significant quantities of chlamydospores during normal vegetative growth (for example Laetiporus sulphureus) or B) inducement of chlamydospore production via stimuli in species that produce chlamydospores under specific conditions (for example many Trametes spp.).

By producing, liberating, and dispersing chlamydospores with the “slurry” inoculation technique described above more points of inoculation are created leading to the following benefits:

1) A reduction in time between inoculation and the fungus fully colonizing the substrate

2) Increased competitiveness with other microorganisms, leading to reduced contamination rates and greater yields

3) Because of 1 and 2 above less spawn is required to inoculate a given mass of substrate, leading to reduced cost.

4) Because the process can utilize a wide range of spawn types, including grain spawn (the current standard for mushroom and fungal cultivation), new spawn production paradigms do not need to be developed. This will allow for faster and more economical implementation at scale.

5) Increased efficacy of other, non-traditional, lignocellulose substrates as spawn (such as agricultural waste including corn stover) that may be less nutritive than traditional grain and sawdust based spawn. By increasing the effectiveness of these non-traditional spawn types more accessible and economic substrates may be used, leading to reduced cost.

The process does not require the cultivation of fruiting bodies and maintains a single genetic individual.

In another embodiment, the process uses a carbon 5 sugar (such as xylose), alone or with a yeast nutrient (such as spent brewer's yeast (SBY)), in combination with the slurry inoculation technique described in the process steps above to 1) reduce or eliminate the need for any treatment that pasteurizes or sterilizes the substrate intended for inoculation, and 2) reduce or eliminate the need for aseptic handling of pasteurized/sterilized substrate during inoculation. This result is a product of A) greater competition with contaminant mold and bacteria spores via distribution of chlamydospores B) the carbon 5 sugar, namely xylose, being generally inaccessible as a carbon source for many bacteria, but accessible to fungi as a carbon source C) the yeast nutrient provides necessary nutrients for better utilization of the carbon 5 sugar by the fungus intended for cultivation (nitrogen, protein, vitamins), D) many fungi produce xylitol (an anti-microbial compound) as a by-product of xylose decomposition further increasing competitiveness with other microbes. As a result of A, B, C, and D above the fungus intended for cultivation is selected for over other organisms, leading to out-competition of ambient microbial bioburden present in the substrate intended for colonization.

Examples from Supporting Research
Correlation of Presence of Chlamydospores with Reduced Colonization and Contamination Rate

Table 1 below shows a side-by-side comparison of a Ganoderma spp. known for producing a significant quantity of chlamydospores and a Trametes spp. that does not produce a functionally significant quantity of chlamydospores. The result below has been routinely repeated and is indicative of chlamydospore rich slurry inoculation.

Procedure: Inoculation of 5 L bags of lignocellulose with grain spawn slurry inoculation per Process Steps above, as well as with the liquid fraction of slurry only. “Control” refers to standard grain inoculation. All bags were incubated until A) discreet lignocellulose particles were fully colonized with a cohesive network of mycelium without the presence of contaminant organisms or B) a contaminant organism (mold or bacteria) was visually apparent.

TABLE 1

| | Grain: H2O slurry | Liquid fraction | Control |
|---|---|---|---|
| Ganoderma spp. | | | |
| % of bags colonized | 100% | 33% | 0% |
| without contaminant | | | |
| Contaminant | NA | Rhizopus | Rhizopus |
| organism | | | |
| Trametes spp | | | |
| % of bags colonized | 0% | 0% | 0% |
| without contaminant | | | |
| Contaminants | Rhizopus | Rhizopus | Rhizopus |
| organism | | | |

### Functional Concentrations of Chlamydospores

Approximately 8,000 chlamydospores/ml slurry inoculum has been found to be the minimum functionally meaningful concentration. Chlamydospore concentrations at this level and lower result in limited benefits over standard inoculation procedures.
Approximately 40,000 to 400,000 chlamydospores/ml slurry inoculum; this has been found to be a functional range, leading to the benefits described above.
Up to approximately 1,500,000 chlamydospores/ml slurry inoculum; this concentration is achieved with ideal optimization per Process Steps.

### Decreased Contamination Rate at Lowered Inoculation Rates

Table 2 below shows the results of a Production scale experiment comparing 5% (by dry weight of substrate intended for inoculation) grain spawn:H₂O slurry inoculum per above process to 17%, 15%, and 12% standard inoculation of solid lignocellulose. Context: during incubation heat conditions unexpectedly increased, leading to significant bacterial contamination.

TABLE 2

| | Total | Number of bags | |
|---|---|---|---|
| | bags | contaminated | Yield |
| 5% slurry inoculation | 32 | 3 | 90.62% |
| 17% standard grain inoculation | 32 | 7 | 78% |
| 15% standard grain inoculation | 34 | 12 | 65% |
| 12% standard grain inoculation | 32 | 14 | 56% |

As shown above, slurry inoculum results in more robust colonization with lesser instances of contamination, even at significantly reduced inoculation rates. This leads to significantly increased yield, protection from unexpected shifts in incubation conditions, and reduced consumption of spawn.

### Increased Growth Rate Over Standard Inoculation Practice

Table 3 below is a comparison of typical growth rates, indicated as number of days from inoculation to full colonization of solid lignocellulose substrate, between standard grain inoculation and grain:H₂O slurry inoculation. Percentages refer to amount of grain used per dry mass of substrate being inoculated.

TABLE 3

| | Days |
|---|---|
| 5% grain: H₂O slurry | 3-4 |
| 1% grain: H₂O slurry | 4 |
| 5% standard inoculation | 6 |
| 17% standard inoculation | 5 |
| 28% standard inoculation | 4-5 |

Inoculation and Colonization of Raw (Non-Sterilized/Pasteurized) Substrate with Non-Aseptic Procedure

FIG. 2 shows non-sterilized/pasteurized lignocellulose successfully inoculated (nonaseptically) and colonized with a given fungus via slurry inoculation with xylose added per 4 of process steps above. Discrete particles of lignocellulose 1 remain exposed due to less dense mycelial growth as compared to the xylose-SBY medium of FIG. 3.

FIG. 3 shows non-sterilized/pasteurized lignocellulose successfully inoculated (nonaseptically) and colonized with a given fungus via slurry inoculation with xylose and spent brewer's yeast (SBY) added per 4 of process steps above.

Slurry inoculation with SBY combined with xylose leads to a denser quality of colonization than xylose alone.

### Alternative Stimuli for the Production of Chlamydospores

FIG. 4 illustrates an SEM imaging of a large cluster of chlamydospores produced by a Daedaleopsis spp. after aging (step 2b per process steps). Chlamydospores at various stages of development are apparent including large mature chlamydospores 2 and small immature chlamydospores 3 as well as hyphae 4 of the mycelium. Chlamydospores are otherwise rare in this species during normal vegetative growth.

FIG. 5 illustrates a light microscope image of ovoid 5 and ellipsoid 6 chlamydospores produced by a Trametes spp. after a period of submergence in water (step 8 per process steps). Chlamydospores are otherwise rare in this species during normal vegetative growth.

Further, soaking (per step 8 of process steps) from 1-7 hours prior to inoculation, has been found to meaningfully increase chlamydospore concentration in slurry inoculum for species that also produce a functionally meaningful concentration of chlamydospores during normal vegetative growth. This is especially dramatic for spawn substrates other than grain (which have sub-optimal nutrition profiles); for example, soaking pre-colonized hemp for a period of 3 hours has been found to result in a more than 4× increase in chlamydospore concentration in slurry inoculum.

### Applications

1) Inoculation process
   a) Increased growth rate, decreased loss to contamination, and reduced inoculation rate result in reduced costs and increased throughput. This would be beneficial to a number of processes including biotechnological and mushroom cultivation practices.
   b) The more economical utilization of other lignocellulose substrates (like corn stover and other agricultural wastes, per “Alternative stimuli for the production of chlamydospores” above) provides for the potential to reduce consumption of foodstuffs (such as millet and rye grain) for fungal spawn.
2) Elimination of pasteurization, sterilization, aseptic technique. Utilization of waste streams.
   a) Nearly all existing commercial and industrial methods of fungal cultivation require heat treatment of the substrate and aseptic technique. The reduction or elimination of these process steps would lead to a significant reduction in cost.
   b) Additional nutrients (carbon 5 sugar and yeast nutrient) can be obtained as waste streams from existing systems; carbon 5 as xylose from biofuel production and yeast nutrient as spent brewer's yeast from the brewing industry.

### EXAMPLE 1

### Macerated Slurry Inoculation

1) Obtain millet grain spawn of Basidiomycete spp.
2) Maintain spawn in refrigeration for at least 24 hours prior to use
3) Combine spawn with water at a rate of 1:12 (w:w, spawn:water)
4) Macerate spawn:water slurry with a homogenizer until all solids have been disbursed into a homogeneous liquid slurry
5) Perform one of the following:
   a) Allow macerated slurry to rest at room temperature for a period of up to 6 hours prior to use
   b) Continue to step 6
6) Prepare a substrate of lignocellulose with 1% Ca and 60% moisture content and heat pasteurize the substrate, allowing to cool to room temperature prior to inoculation
7) Combine macerated slurry inoculum with substrate of step 5, agitating substrate thoroughly to homogeneously distribute inoculum throughout mass of substrate
8) Incubate at room temperature until mycelium grows throughout and binds together discrete particles of the lignocellulose.

### EXAMPLE 2

### Agitated Slurry Inoculation

1) Perform steps 1 and 2 of Example 1
2) By hand, or with mechanical stirrer, swirl/stir spawn:water slurry aggressively for a period of at least 60 seconds
3) Perform steps 5-8 of Example 1

### EXAMPLE 3

### Separation of Grain and Liquid Fractions of Slurry Inoculum

1) Perform steps 1-3 of Example 1
2) Perform step 4 of Example 1 or step 2 of Example 2
3) Perform steps 5 and 6 of Example 1
4) Pour or pump slurry inoculum through a sieve to isolate solid fraction of slurry from Liquid fraction
5) Combine one or both of liquid and solid fractions with substrate
6) Perform step 8 of Example 1

### EXAMPLE 4

### Optimization of Chlamydospore Concentration in Spawn Prior to Slurry Production

1) Perform step 1 of Example 1
2) Perform any combination of the following
   a) Place spawn in bag without air exchange interfaces/aeration for 24 hours prior to use
   b) Incubate spawn for 1-4 weeks prior to use, agitating periodically to maintain discrete particles
   c) Place spawn in refrigeration for at least 24 hours prior to use
   d) Place spawn in freezer below 32 F for at least 24 hours prior to use
   e) Place spawn in incubator between 90-180 F for at least 1 hour
3) Perform steps 1-8 of Example 1

### EXAMPLE 5

### Preparation and Inoculation of Raw Substrate with Spawn:Xylose-Spent Brewer'S Yeast Slurry

1) Perform steps 1 and 2 of Example 1
2) Combine water with 10 g/L xylose and 50 ml/L spent brewer's yeast (SBY) and stir to dissolve xylose
3) Combine spawn with water:xylose:SBY at a rate of 1:12 (w:w)
4) Perform step 4 of Example 1 or step 2 of Example 2
5) Perform step 5 of Example 1
6) Prepare corn stover with 1% Ca and 60% moisture content, do not pasteurize substrate
7) Perform steps 7 and 8 of Example 1

The invention thus provides a process for optimizing concentrations and distribution of chlamydospores during inoculation of solid or liquid substrate with a given fungus, namely with Basidiomycete species within the order Polyporales that produce significant quantities of chlamydospores during primary vegetative growth or under specific environmental or nutritive conditions.

The invention provides a process that allows asexual chlamydospores to be derived from a mother vegetative mycelium and that allows both the mother mycelium and asexual chlamydospores to be used as an inoculant concurrently. That is to say, the “mother mycelium” refers to the mycelium grown on the grain/inoculum particles while the chlamydospores are produced on and released from the “mother mycelium”.

The invention further provides a process that leverages the dispersal and germination of chlamydospores during common inoculation procedures (i.e. dispersal of spawn consisting of particles of substrate pre-colonized with a fungus through virgin substrate).

The invention further provides a process that obtains an increased growth rate of fungus through a substrate as compared to traditional inoculation techniques via distribution and germination of chlamydospores while obtaining a higher concentration of chlamydospores in a mother mycelium prior to inoculation.

The invention also allows the process to use non-traditional lignocellulose substrates as spawn in the production of chlamydospores.

What is claimed is:

1. A process for the production of a chlamydospore rich slurry inoculum comprising the steps of
obtaining spawn consisting of a substrate colonized with a desired Basidiomycete fungus capable of producing chlamydospores during vegetative growth;
increasing the chlamydospore production and content in said spawn; and
thereafter adding water to the spawn at rate of at least 1:6 spawn:water to obtain a slurry inoculum.

2. A process as set forth in claim 1 wherein said substrate is a grain.

3. A process as set forth in claim 2 wherein said grain is selected from the group consisting of cereal grain, millet and rye.

4. A process as set forth in claim 1 wherein said substrate is a lignocellulose.

5. A process as set forth in claim 4 wherein said lignocellulose is selected from the group consisting of cereal straw, corn stover and hardwood sawdust.

6. A process as set forth in claim 1 wherein said step of increasing the chlamydospore production and content in said spawn includes at least one of
placing said spawn under respiratory stress in a container without gas exchange interfaces to stimulate chlamydospore formation,
incubating said spawn for a period of time sufficient to stimulate chlamydospores formation under nutritive stress,
refrigerating said spawn between 32-50° F. for at least 24 hours,
freezing said spawn below 32° F. for at least 24 hours, and
heating said spawn to between 90° F. and 180° F. for at least one hour.

7. A process as set forth in claim 1 further comprising the step of adding a nutrient to said slurry inoculum.

8. A process as set forth in claim 7 wherein said added nutrient is selected from the group consisting of
no more than 5 g/L of a carbon 6 monosaccharide or oligosaccharide,
5-20 g/L carbon 5 monosaccharide, and
at least 50 ml/L yeast nutrient.

9. A process as set forth in claim 8 wherein carbon 6 said monosaccharide or oligosaccharide is one of glucose and maltodextrin.

10. A process as set forth in claim 8 wherein said carbon 5 monosaccharide is xylose.

11. A process as set forth in claim 8 wherein said yeast nutrient is spent brewer's yeast.

12. A process as set forth in claim 1 further comprising the step of agitating said slurry to release chlamydospores from a solid spawn fraction of said slurry and to populate a water fraction of said slurry with said released chlamydospores.

13. A process as set forth in claim 12 further comprising the step of thereafter soaking said slurry for a time sufficient to further stimulate production of chlamydospores via water shock and obtain a chlamydospore rich slurry.

14. A process as set forth in claim 13 further comprising the step of separating said chlamydospore rich slurry inoculum into a solid fraction separate from a liquid fraction.

15. A process as set forth in claim 1 further comprising the step of macerating said slurry to homogeneously distribute chlamydospores in a water fraction of said slurry.

16. A process as set forth in claim 15 further comprising the step of thereafter soaking said slurry for a time sufficient to further stimulate production of chlamydospores via water shock and obtain a chlamydospore rich slurry

17. A process as set forth in claim 16 further comprising the step of separating said chlamydospore rich slurry inoculum into a solid fraction separate from a liquid fraction.

18. A process comprising the steps of
obtaining a millet grain spawn of Basidiomycete spp.;
refrigerating the obtained spawn for at least 24 hours;
thereafter combining the refrigerated spawn with water at a rate of 1:12 spawn:water to obtain a slurry;
macerating the spawn:water slurry to form a homogeneous liquid slurry inoculum;
heat pasteurizing a substrate of lignocellulose with 1% Ca and 60% moisture content;
cooling the pasteurized substrate to room temperature;
thereafter combining the macerated slurry inoculum with the cooled substrate to homogeneously distribute said inoculum throughout said substrate and form a homogeneous mixture thereof; and
incubating said mixture at room temperature for a time sufficient for mycelium to grow throughout said mixture and to bind together discrete particles of lignocellulose.

19. A process as set forth in claim 18 wherein the macerated slurry is allowed to rest at room temperature for a period of up to 6 hours prior to combining with said corn stover.

20. A process comprising the steps of
obtaining a millet grain spawn of Basidiomycete spp.;
refrigerating the obtained spawn for at least 24 hours;
combining water with 10 g/L xylose and 50 ml/L spent brewer's yeast (SBY) while stirring to dissolve said xylose and obtain a water solution;
thereafter combining the refrigerated spawn with said water-xylose-SBY solution at a rate of 1:12 spawn:water to obtain a slurry;
macerating said slurry to form a homogeneous liquid slurry inoculum;
obtaining a substrate of lignocellulose with 1% Ca and 60% moisture content;
thereafter combining the slurry inoculum with said substrate to homogeneously distribute said inoculum throughout said substrate and form a homogeneous mixture thereof; and
incubating said mixture at room temperature for a time sufficient for mycelium to grow throughout said mixture and to bind together discrete particles of lignocellulose.

21. A process as set forth in claim 20 wherein the macerated slurry is allowed to rest at room temperature for a period of up to 6 hours prior to combining with said corn stover.
